(19) United States
(12) Patent Application Publication
(10) Pub. No.: US 2016/0235650 A1
(43) Pub. Date: Aug. 18, 2016

(54) Cosmetic Formulation Incorporating a UV-Triggered Self-Healing Material

(71) Applicant: AVON PRODUCTS, INC, Suffern, NY (US)

(72) Inventor: Claude C. Simard, Sparrowbush, NY (US)

(21) Appl. No.: 14/379,398

(22) PCT Filed: Feb. 26, 2014
(86) PCT No.: PCT/US14/18695

§ 371 Date: Aug. 18, 2014

Related U.S. Application Data
(60) Provisional application No. 61/789,181, filed on Mar. 15, 2013.

(51) Int. Cl. A61K 8/87; A61Q 1/12; A61Q 1/10; A61Q 1/08; A61Q 19/08; A61Q 1/06; A61Q 3/02; A61Q 5/12; A61Q 5/02; A61Q 19/00; A61Q 17/04; A61Q 1/04

(52) U.S. Cl. CPC A61K 8/87; A61Q 17/04; A61Q 1/12; A61Q 1/10; A61Q 1/08; A61Q 1/04; A61Q 1/06; A61Q 3/02; A61Q 5/12; A61Q 5/02; A61Q 19/00; A61Q 19/08

(57) ABSTRACT

A cosmetic composition for treating hair, nails and skin, including the lips, is provided, which composition comprises a UV activated self-repair film forming material, for example, a polymer, such as a polyurethane polymer, containing a polysaccharide substituted with a UV active oxetane, or a metallo-supramolecular film-former capable of reforming polymeric like chains via metal ligand complexation.

### CROSS-REFERENCE TO RELATED APPLICATIONS

This application claims priority benefit, under the national stage entry under 35 U.S.C. 371 of International Application No. PCT/US14/18695, filed on Feb. 26, 2014 the contents of which application are hereby incorporated by reference in their entirety. This patent application claims priority to U.S. Patent Application Ser. No. 61/789,181 filed on Mar. 15, 2013. The entirety of the aforementioned application is incorporated herein in its entirety by reference.

### FIELD OF THE INVENTION

The present invention provides a cosmetic composition for treating hair, nails and skin, including the lips, the composition comprising a UV activated self-repair film forming material, for example, a polymer, such as a polyurethane polymer, containing a polysaccharide substituted with a UV active oxetane, or a metallo-supramolecular film-former capable of reforming polymeric chains via metal ligand complexation.

### BACKGROUND OF THE INVENTION

Long lasting cosmetic compositions for hair, skin, nails and lips that maintain their desired properties and appearance without frequent reapplication are desirable. It has become increasingly common, in order to overcome problems such as migration, transfer, cracking and other causes of wear or loss of cosmetic effect, to incorporate film formers, e.g., natural or synthetic polymers, into such compositions. Such compositions, once applied, can be left on one's hair, nails, lips, facial skin and body skin for extended periods. However, with this increased wear time additional failure modes for the cosmetic have become apparent. For example, the films once formed are subject to wear and damage resulting in defects such as micro-cracks or scratches that can destroy the integrity of the film and lead to loss of desired cosmetic properties.

Ghosh and Urban (Ghosh B. and Urban M. W., Self-Repairing Oxetane-Substituted Chitosan Polyurethane Networks, Science Vol. 323 No. 5920 pp. 1458-1460 (incorporated by reference in its entirety) and US Patent Application Serial No. US 2010/0266784) reported a self-repairing oxetane-substituted chitosan polyurethane network which upon exposure to UV exhibited self repairing characteristics. These materials were able to repair themselves within one hour. Burnworth M. et al. (Nature 472, 224-227) (incorporated by reference herein in its entirety) reported optically healable supramolecular polymers, wherein oligomeric materials utilize absorbing end groups which can coordinate with a transition metal or lanthanide metal to form polymeric structures, which when damaged by cleavage of a chain, can delink at the metal complexing site due to absorbed light, reorganize, and then relink in a manner reestablishing the polymer chain. For example, an ethylene/butylene co-oligomer core was reacted at the ends of the oligomeric chain to incorporate 2,6-bis(1-methylbenzamidazoyl)pyridine groups, which were then complexed to a metal such as zinc to provide a polymer which can reform broken polymer chains when exposed to UV light.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

BRIEF SUMMARY OF THE INVENTION

The present invention provides more robust cosmetic film compositions by incorporating into the composition self healing film forming polymers containing light activated self healing systems.

The invention provides cosmetic compositions comprising one or more common cosmetic actives or adjuvant and a UV activated self-repair material, for example a polymeric material such as a film-forming polymer. The cosmetic compositions of the invention include, for example, compositions applied to the face, lips, nails, hair, eyelashes, eyelids, other body parts, false eyelashes, wigs, fake nails, etc.

The UV activated self repair material, typically a film forming material, is a compound such as an organic polymer or a supramolecular polymeric material such as a material comprising oligomeric portions linked together through complexation of metals to light absorbing ligands attached to the ends of the oligomer chains. When damaged, for example, when the polymeric material is cleaved, the self healing material can reform chemical bonds to reestablish the integrity of the polymeric structure through a process that is initiated by absorption of light, typically UV light. Due to the presence of UV light in sunlight and many artificial light sources, there is no special treatment required to initiate the self-healing process.

Compositions of the invention can be used for almost any cosmetic purpose and include, for example, nail polish, lipsticks, lip gloss, face make-up, mascaras, eye shadow, eye liner, skin creams, lotions, hair products, skin protections, such as sun blocks, anti aging treatments, and thus may contain any suitable cosmetic ingredient, e.g., colorant, fragrance, moisturizer, pH adjuster, filler, emulsifying agent, emollient, sunscreen, retinoid, anti-acne agent, skin protectant and the like.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, and some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

As used herein, “% by weight” or “% wt.” refers to the weight percent of a component in relation to the total weight of the composition (i.e., including any carriers, vehicles, solvents, fillers, or other components added before application to the skin) unless otherwise provided.

Whenever a term is identified by reference to a range, the range will be understood to explicitly disclose every element thereof. As a non-limiting example, C₁₋₄ alkyl will be understood to refer to and disclose C₁, C₂, C₃, or C₄ alkyl.

Where two or more substituents are referred to as being “independently selected from” a group of enumerated alternatives, it is meant that each substituent can be any element of that group, independent of the identity of the other substituents.

As used herein, all terms are intended to have their ordinary meaning in the art unless specifically defined.

As used herein, the term “film-forming” polymer is understood to mean a polymer capable of forming on its own, or in the presence of a film-forming aid, a continuous and adherent film on a support, such as on skin, hair, nails or other keratin materials.

As used herein, the term “pigments” is understood to mean white or colored, inorganic or organic particles that are insoluble in the liquid organic phase, intended to color and/or opacify the first composition.

As used herein, the term “pearlescent pigments” is understood to mean iridescent particles produced for instance, by certain mollusks in their shell or alternatively synthesized, which are insoluble in the medium of the first composition.

As used herein, the term “dyes” is understood to mean generally organic compounds that are soluble in fatty substances such as oils or in an aqueous-alcoholic phase.

As used herein, the term “filler” means any material selected from inorganic or organic, lamellar or non-lamellar mineral or synthetic, spherical or oblong fillers that are chemically inert in the composition.

All references to median or mean particle sizes herein are on a volume basis. All amounts provided in terms of weight percentage are relative to the entire composition unless otherwise stated.

The cosmetic compositions of the invention comprise the self healing material in amounts of from about 0.1 to about 60% by weight, for example from about 1 to about 40%, for example from about 2 to about 30% and in some embodiments from about 5% to about 20% by weight of the cosmetic product, depending upon the desired end use of the cosmetic composition. For example, if the desired end use of the cosmetic composition is a lip color composition, such desired properties include ease of application, length of drying time, aesthetic feel to the user's lips, and the like. Thus in a lip color composition, the amount of the film-forming polymer, including the self-healing polymer of the invention is from about 10 to about 30% by weight.

In many embodiments the self-healing material is a polymer, for example a film forming polymer. Other polymers or film formers may of course also be present in the composition.

In one embodiment, the self-healing material is a polymer which comprises a condensation polymer, such as a polyester, polyamide, or polyurethane, wherein one of the components used to make the polymer comprises a 4-8 membered oxygen containing ring, for example a four membered oxetane ring. Examples of such polymers can be found, for example, in US 2010/0266784. The condensation polymers are prepared using well known processes.

In the case of polyurethanes, for example, a polyol is reacted with a polyisocyanate, which may also be further reacted with a cross linking agent. The polyol may be a simple diol, e.g., propane diol, but in many instances a higher molecular weight polyol is used, such as a polyalkylene glycol, or a polyester polyol, which is essentially a polyester oligomer or polymer end capped with hydroxyl groups. In some cases, more than one polyol or polyisocyanate is used. A wide variety of polyols and polyisocyantes useful for preparing polyurethanes are known.

When the self-healing material of the invention is a polyurethane, one of the reaction components comprises the above mentioned oxygen containing ring, typically a four membered oxetane ring. Frequently, the oxetane ring is found as part of a derivatized polysaccharide. Examples of certain self-healing polyurethane polymers of the invention, as well as oxetane containing polysaccharides, polyols and polyisocyanates useful in preparing them, can be found in US 2010/0266784.

For example, a naturally occurring polysaccharide, such as chitosan, pectin or, heparin, e.g., chitosan, is reacted with an oxetane compound to prepare an oxetane comprising reactant useful for reacting into the polyurethane. For example, chitosan and a chloromethyl oxetane derivative can be reacted to provide an oxetane substituted chitosan derivative of the invention which is then reacted with a polyisocyanate in the formation of the desired polyurethane polymer, e.g., a chitosan derivative of formula (I)

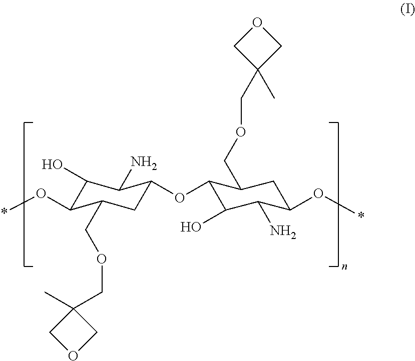

In this example, the oxetane is 3,3 dimethyloxetane.

Typically, polyols, including simple alkane diols, polyalkylene diols and/or polyester diols, are reacted with the polyisocyanate either along with the oxetane containing chitosan or in a separate reaction before or after the isocyanate/polysaccharide reaction. In other embodiments, a thiol containing component can be used in place of the polyol, or along with a polyol to prepare the self-healing polymer. For example, the present invention may comprise self-healing polymers of formula

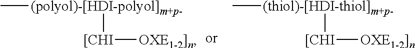

wherein CHI-OXE₁₋₂ represents chitosan substituted by oxetane components, e.g. as shown in figure (I), HDI represents a polyisocyanate, m=number of moles of an isocyanate used to produce the composition; p=number of moles of the polyol or thiol component used to produce the composition, and n=number of moles of a chitosan compound used to produce the composition. Values of m, p and n will vary widely depending on the intended use for the polymer as understood by one skilled in the art as set forth in US 2010/0266784, e.g. m ranges from 3-450, p ranges from 0-540 and n ranges from 5-650.

In one embodiment of the invention the self healing film forming polymer comprises a reaction product of: a) a polyol or a thiol; b) an isocyanate; and c) an oxetane substituted polysaccharide.

Polyisocyanates useful in the invention can include methylenediisocyanate (MDI); hexamethylenediisocyanate (HDI); isophorenediisocyanate (IPDI); toluenediisocyanate (TDI); 2,4-toluene diisocyanate; 2,6-toluene diisocyanate; 2,2′-methylenediphenylene diisocyanate; 2,4′-methylenediphenylene diisocyanate; 4,41-methylenediphenylene diisocyanate; polyphenylene polymethylene polyisocyanate; saturated 2,4-methylcyclohexane diisocyanate, saturated 2,6-methylcyclohexane diisocyanate; 2,2′-methylene dicyclohexylene diisocyanate; 2,4′-methylene dicyclohexylene diisocyanate; 4,4′-methylene dicyclohexylene diisocyanate; isophorone diisocyanate; 1,4-diisocyanatobutane; 1,5-diisocyanatopentane; 1,6-diisocyanatohexane; 1,4-cyclohexane diisocyanate; isomeric mixtures thereof; or combinations thereof.

Polyols of the invention include ethylene glycol, diethylene glycol, triethylene glycol, tetraethylene glycol, polyethylene glycol, propylene glycol, dipropylene glycol, polypropylene glycol, 1,4-butanediol, 1,6-hexanediol, polytetramethylene glycol, polyesterdiol, derivatives thereof, or combinations thereof.

Thiols useful in the invention include pentaerythritol tetrakis(3-mercaptoproprionate) (PET3MP), trifunctional thiols, tetrafunctional thiols, thiol esters, thiol acrylates, or combinations thereof.

It is also possible to incorporate silicone-containing polyols such as found in US 2005/0238611 and U.S. Pat. No. 8,128,919 into the polyurethane polymer above.

In another embodiment, the self-healing material is a metallo-supramolecular polymer such as found in Nature 472, 224-227 (2011). That is, polymers formed when oligomers containing light absorbing metal chelating ligand end groups are reacted with a chelating metal to produce a metal linked polymer. Typically, an excess of ligand is present in the polymer mixture so that uncomplexed ligand remains. When an oligomer chain is ruptured or cleaved, the polymer chain is shortened and the morphology of the polymer is altered. The metal and ligand are selected so that upon absorption of light, e.g., UV light, the metal can disengage from the ligand, which changes the morphology of the polymer and greatly reduces the local viscosity. The metal can then chelate to different sets of ligands to reform a sufficiently large polymer chain to re-establishes the local viscosity, morphology and intermolecular forces necessary to recreate the properties of the original polymer.

Oligomers useful in the invention are chosen so that they can be functionalized to bind the desired ligand to the oligomer ends. In the cited art, an ethylene/butylene co-oligomer bearing primary hydroxyl substituents was reacted with 4-hydroxy-2,6-bis(1-methylbenzamidazoyl)pyridine under Mitsunubu reaction conditions, i.e., triphenyl phosphine and diethyl azodicarboxylate, to form the ligand substituted oligomer. Addition of Zn(NTf₂)₂, i.e., zinc di[bis(trifluoromethanesulfonyl)imide, generated the zinc linked supramolecular polymer species.

In one particular embodiment the self healing film forming polymer is prepared by treating a 2,6-bis(1-methyl benzamidazoyl)pyridine substituted ethylene/butylene co-oligomer with a Zn salt, e.g., Zn(NTf₂)₂ or a La salt, e.g. La(NTf₂)₃.

Other oligomeric species can be incorporated as the oligomeric core of these polymers, e.g., oligomers formed from other olefins, polyalkylene glycols, polysilanols, etc., the proviso being of course that functional groups of the oligomer do not interfere with the metal/ligand chelation, and that functionality does exist in the oligomer to provide for attachment of the chosen ligand.

In theory other light absorbing ligands may be used provided that the chelation with a metal can occur with the appropriate strength so that the metal can delink and reform the polymer upon exposure to light, and the intermolecular forces in the complexed supramolecule are favorable for generating a coherent film. Likewise other metals are possible provided the same conditions are met.

When choosing any of the materials for use in the present cosmetic compositions, care must be taken to assure compatibility, and obviously the materials must be safe and non-irritating to humans.

As is common in the field the molecular weights of the self-healing polymers can vary widely depending on the final use.

Typically, the self-healing material is a UV activated, self-healing, polymeric, film former. As such, it is possible to use the inventive polymers in many known compositions as “drop in” replacements or substitutes for currently used film formers. In certain cases it may be desirable to replace a portion of the currently used film forming with the inventive self-healing materials. As such, the compositions of the invention may comprise the self healing polymers and almost any cosmetic ingredients.

The cosmetic compositions of the invention will comprise other active and inactive ingredients, well known in the field including, but not limited to, cosmetically acceptable carriers, oils, waxes, sterols, amino acids, moisturizers, powders, ultraviolet absorbents, colorants (including pigments and/or dyes) pH adjusters, perfumes and other fragrances, essential oils, flavor oils, cosmetic active ingredients, vitamins, essential fatty acids, sphingolipids, self-tanning compounds such as DHA, excipients, fillers, emulsifying agents, antioxidants, surfactants, additional film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, skin cooling compounds, skin protectants, skin penetration enhancers, exfolients, lubricants, staining agents, depigmenting agents, hypopigmenting agents, preservatives, stabilizers, pharmaceutical agents, photostabilizing agents, spherical powders etc.

Such ingredients are well known in the art and examples of specific materials and typical concentrations can be found, for example, in the US patents and published applications cited above, for example, U.S. Pat. No. 5,948,393; U.S. Pat. No. 6,471,983; U.S. Pat. No. 4,122,023; U.S. Pat. No. 6,312,672; U.S. Pat. No. 6,258,348; U.S. Pat. No. 8,128,919; U.S. Pat. No. 8,133,478; US 2005/0238611; US 20050276779 and US 20090285866, the relevant portions of which are included herein by reference.

There are a great number of other ingredients approved for use in the cosmetic art that may be used in compositions of the present invention. For example approved ingredients can be found in reference books such as the CTFA Cosmetic Ingredient Dictionary & Handbook, Tenth Edition, The Cosmetic, Toiletries, and Fragrance Association, Inc. 2004 and The Handbook of Cosmetic Science and Technology, 1st Ed. Knowlton & Pearce (Elsevier 1993).

The pigments may be white or colored and inorganic or organic. Inorganic pigments include titanium dioxide, zirconium dioxide and cerium dioxide, as well as zinc oxide, iron oxide, chromium oxide and ferric blue. Organic pigments also include carbon black and barium, strontium, calcium or aluminum lakes. The pearlescent pigments include mica coated with titanium oxide, with iron oxide, with natural pigment or with bismuth oxychloride, and colored titanium mica.

It is within the skill in the art to choose coloring agents and combinations of coloring agents to produce a desired color. Suitable coloring agents, including pigments, lakes, and dyes, are well known in the art and are disclosed in the C.T.F.A., International Cosmetic Ingredient Dictionary and Handbook, 10.sup.th Edition, 2004. Organic pigments include, but are not limited to, for example, FD&C dyes, D&C dyes, including D&C Red, Nos. 2, 5, 6, 7, 10, 11, 12, 13, 30 and 34, D&C Yellow No. 5, Blue No. 1, Violet No. 2 and the like. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Other suitable colorants include ultramarine blue (i.e., sodium aluminum silicate containing sulfur). Prussian blue, manganese violet, bismuth oxychloride, talc, mica, sercite, magnesium carbonate, calcium carbonate, magnesium silicate, aluminum magnesium silicate, silica, titanated mica, iron oxide titanated mica, and the like. The colorants may be surface modified with, for example, fluoropolymers, to adjust one or more characteristics of the colorant. Suitable pearling pigments include without limitation bismuth oxychloride, guanine and titanium composite materials containing, as a titanium component, titanium dioxide, titanium lower oxides or titanium oxynitride. Other suitable pearlescent materials typically are pigments or layers of titanium dioxide on a substrate such as mica, polyethylene terephthalate, bismuth oxychloride, aluminum oxide, calcium borosilicate, synthetic flourophlogopite (synthetic mica), silica, acrylates copolymer, methyl methacrylate, and the like.

The pearlescent pigments can be chosen from white pearlescent pigments, such as mica covered with titanium or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type.

The compositions of the present invention may contain a retinoid or an alpha hydroxy acid, e.g. glycolic acid and lactic acid, for enhancing skin appearance benefits, including regulating signs of skin aging, more especially wrinkles, lines, and pores. The retinoid may be included as the substantially pure material, or as an extract obtained by suitable physical and/or chemical isolation from natural (e.g., plant) sources. The compositions may contain from about 0.005% to about 2%, more preferably 0.01% to about 2%, retinoid or from 0.1% to about 6.0% alpha hydroxy acid.

The compositions of the present invention may contain an anti-oxidant/radical scavenger. The anti-oxidant/radical scavenger is especially useful for providing protection against UV radiation and against other environmental agents which can cause skin damage. A safe and effective amount of an anti-oxidant/radical scavenger may be added to the compositions of the subject invention, preferably from about 0.1% to about 10%, e.g., from about 1% to about 5%, of the composition. Anti-oxidants/radical scavengers such as ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid, gallic acid and its alkyl esters, especially propyl gallate, uric acid and its salts and alkyl esters, sorbic acid and its salts, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used.

Waxes, fats, and emollients useful in the present compositions include natural animal and vegetable fats and oils, and semi-synthetic fats and oils, examples of which include avocado oil, linseed oil, almond oil, Chinese wax, perilla oil, olive oil, cacao butter, kapok wax, kaya oil, carnauba wax, liver oil, candellila wax, beef tallow, beef foot oil, beef bone fat, hydrogenated beef tallow, apricot kernel oil, spermaceti, hydrogenated oil, wheat germ oil, sesame oil, rice germ oil, rice bran oil, sugar cane wax, sasanqua oil, safflower oil, shea butter, Chinese tung oil, cinnamon oil, jojoba wax, shellac wax, turtle oil, soybean oil, tea seed oil, tsubaki oil, evening primrose oil, corn oil, lard, rape seed oil, Japanese tung oil, rice-bran wax, germ oil, horse fat, persic oil, palm oil, palm kernel oil, castor oil, hydrogenated castor oil, caster oil fatty acid methyl ester, sunflower oil, grape seed oil, bayberry wax, jojoba oil, macadamia nut oil, bees wax, mink oil, cottonseed oil, cotton wax, Japan wax, haze kernel oil, montan wax, coconut oil, hydrogenated coconut oil, tricoconut oil fatty acid glyceride, mutton-tallow, peanut oil, lanolin, liquid lanolin, reduced lanolin, lanolin alcohol, hard lanolin, lanolin acetate, lanolin fatty acid isopropyl, hexyl laurate, POE lanolin alcohol ether, POE lanolin alcohol acetate, lanolin fatty acid polyethylene glycol, POE hydrogenated lanolin alcohol ether, and egg yolk oil. Additionally, the term “POE” as used herein stands for polyoxyethylene. Other waxes include linear hydrocarbon-based waxes, ethylene polymers, linear paraffin waxes, silicone wax, and long-chain linear alcohols.

Among the oils that may be used include esters derived from long chain acids or alcohols for instance palmitates, adipates and benzoates, for example diisopropyl adipate, linear, branched and/or cyclic, optionally volatile, alkanes, for instance paraffin oils, petroleum jelly or hydrogenated polyisobutylene, isododecane volatile isoparaffins. silicone oils such as polydimethylsiloxanes and polymethylphenylsiloxanes, optionally substituted with fluorinated groups, or with functional groups such as hydroxyl, thiol and/or amine groups, and volatile silicone oils.

A filler may be added either for adjusting its flowability or improving the mechanical strength of the obtained cross-linked silicone particles. Examples of suitable fillers are precipitated silica, fumed silica, baked silica, mica, talc, fumed titanium oxide, kaolin, or a similar reinforcing filler; natural mother-of-pearl, boron nitride, crushed quartz, diatomaceous earth, alumino silicic acid, ferrous oxide, zinc oxide, calcium oxide, precipitated calcium carbonate, magnesium carbonate and hydrocarbonate, polyamide powders, powders of tetrafluoroethylene polymers, starch, hollow polymer microspheres, acrylic polymers particles and mixtures thereof, or a similar non-reinforcing filler. The surfaces of these fillers can be treated with hexamethylsiloxane, trimethylchlorosilane, polydimethylsiloxane, polymethylhydridosiloxane or similar organosilicone compounds.

The solid pigments, pearlescent agents or fillers may be dispersed in the liquid fatty phase of the composition in the presence of at least one dispersing agent. The at least one dispersing agent can serve to protect the dispersed particles against their agglomeration or flocculation. The dispersing agent may be a surfactant, an oligomer, a polymer or a mixture of several of them, bearing one or more functionalities having a high affinity for the surface of the particles to be dispersed. For instance, they may become physically or chemically attached to the surface of the pigments. These dispersants additionally have at least one functional group that is compatible or soluble in the continuous medium. By way of non-limiting example, use may be made of the esters of 12-hydroxystearic acid and a polyol such as glycerol, diglycerin, such as the stearate of poly(12-hydroxystearic) acid, polyglycerol-2 dipolyhydroxystearate, or polyhydroxystearic acid. Other non-limiting dispersants include quaternary ammonium derivatives of polycondensed fatty acids and mixtures of poly dimethylsiloxane/oxypropylene.

At least one additional film-forming polymer may be used in the composition of the present invention may include, but are not limited to, synthetic polymers, natural polymers and mixtures thereof. Film-forming polymers that may be mentioned in particular include acrylic polymers, polyurethanes, polyesters, polyamides, polyurea, and cellulose-based polymers, such as nitrocellulose.

Active ingredients may also include chemical sun filters useful in the cosmetic field including any UVA and UVB filter useful in the cosmetic field including mixtures thereof and blends with physical filters. Physical filters include, but are not limited to, titanium, zinc, iron, zirconium, cerium oxides or metal oxides, or mixtures thereof.

The composition will often comprise water and may also contain organic solvents such as esters, ethers, alcohols, ketones, glycols, cyclic ethers, alkanes, alkyl sulphoxides heterocyclic compounds such as tetrahydrofuran, propylene carbonate and the like.

The procedure for manufacture of the compositions according to the invention do not differ in any way form the procedures conventionally used in the cosmetics field and are entirely known to the specialist. These procedures consist in mixing the different constituents of the composition, preferably after heating, then in pouring them to produce the desired shape.

The compositions according to the invention may take the form of sticks or of flexible or poured pastes, or a viscous liquid, depending upon the desired end use of the cosmetic composition according to the present invention.

The cosmetic compositions may be in the form of cosmetically acceptable vehicles, such as, but not limited to, a lotion, a cream, a paste, an ointment, a soft paste, a liquid (e.g. suspension or solution), a salve, a wax, a solid that has been cast or molded, such as a stick or a dish, or a compacted solid, a powder, a mousse, ointment, spray, milk, foam, balm, aerosol an oily or aqueous solution, an oily or aqueous gel, an oil-in-water or water-in-oil emulsion, a multiple emulsion etc.

The cosmetic composition may include skin care cosmetics such as a skin lotion, skin milk, skin cream, gel, skin softener, foundation, foundation primer base, blush, make-up rouge, lipstick, lip gloss, lip balm, eye shadow, eye liner, eye pencil, mascara, nail enamel, concealer, sunscreen, anti-sun product, skin coloring product such as a self-tanning product, semi-permanent make-up product (tattoo). The composition may also be a hair care product for thickening, conditioning, holding or shaping the hair, and the like.

### EXAMPLES

The following examples are meant to demonstrate certain potential aspects of the invention in a non-limiting fashion.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description, Examples, and figure. Such modifications are also intended to fall within the scope of the appended claims. All publications cited herein are incorporated by reference in their entirety.

Specific cosmetic compositions of the invention are shown below. The Oxetane-Substituted Chitosan Polyurethane may be prepared from hexamethylenediisocyanate, polyethylene glycol and dimethyl oxetane substituted chitosan polysaccharide according to US 2010/0266784. The Metallo-Supramolecular Polymer may be the 2,6-bis(1-methyl benzamidazoyl)pyridine substituted ethylene/butylene co-oligomer treated with of Zn(NTf₂)₂ described in Nature 472, 224-227 (2011).

Nail Polish

| Ingredients | Weight % | Weight % |
|---|---|---|
| Ethyl Acetate | 20.0-35.0 | 20.0-35.0 |
| Butyl Acetate | 12.0-30.0 | 12.0-30.0 |
| Nitrocellulose (30% IPA) | 2.0-10.0 | 2.0-10.0 |
| Adipic acid/Neopentyl Glycol/Trimellitic | 2.0-8.0 | 2.0-8.0 |
| Anhydride Copolymer | | |
| Oxetane-Substituted Chitosan Polyurethane | 5.0-20.0 | — |
| Metallo-Supramolecular Polymer | — | 5.0-20.0 |
| Tosylamide/Epoxy Resin | 2.0-10.0 | 2.0-10.0 |
| Isopropyl Alcohol | 5.0-15.0 | 5.0-15.0 |
| Triphenyl Phosphate | 1.0-5.0 | 1.0-5.0 |
| Trimethyl Pentanyl Diisobutyrate | 1.0-5.0 | 1.0-5.0 |
| Stearalkonium Bentonite | 0.5-2.0 | 0.5-2.0 |
| Titanium Dioxide | 0.7 | 0.7 |
| D&C Red No. 7 Calcium Lake | 0.7 | 0.7 |
| D&D Red No. 34 Calcium Lake | 0.3 | 0.3 |

Long Wear Lipstick

| Ingredients | Weight % | Weight % |
|---|---|---|
| Isododecane | 3.0-10.0 | 3.0-10.0 |
| Cyclopentasiloxane | 7.0-15.0 | 7.0-15.0 |
| Phenyl Trimethicone | 3.0-8.0 | 3.0-8.0 |
| Isononyl Isononanoate | 5.0-15.0 | 5.0-15.0 |
| Octyl Methoxycinnamate | 5.0 | 5.0 |
| Oxetane-Substituted Chitosan Polyurethane | 5.0-15.0 | — |
| Metallo-Supramolecular Polymer | — | 5.0-15.0 |
| PVP/Hexadecene Copolymer | 0.5-4.0 | 0.5-4.0 |
| Microcrystalline Wax | 2.0-8.0 | 2.0-8.0 |
| Ozokerite 170 | 3.0-8.0 | 3.0-8.0 |
| Paraffin | 1.0-5.0 | 1.0-5.0 |
| Beeswax | 1.0-5.0 | 1.0-5.0 |
| Lanolin | 2.0-10.0 | 2.0-10.0 |
| Diisoterayl Malate | 1.0-5.0 | 1.0-5.0 |
| Tocopheryl Acetate | 0.05 | 0.05 |
| Nylon Powder | 1.0-5.0 | 1.0-5.0 |
| Pearls | 0.0-10.0 | 0.0-10.0 |
| D&C Red No. 7 Ca. Lake (ITT treated) | 1.5 | 1.5 |
| D&C Red No. 6 Bar. Lake (ITT Treated) | 1.5 | 1.5 |
| Red Iron Oxide | 0.65 | 0.65 |
| Batium Sulfate | 0.5-5.0 | 0.5-5.0 |
| Mica | 0.5-5.0 | 0.5-5.0 |
| Fragrance | 0.2 | 0.2 |

Lip Gloss

| Ingredients | Weight % | Weight % |
|---|---|---|
| Polybutene | 15.0-28.0 | 15.0-28.0 |
| Lanolin | 4.0-12.0 | 4.0-12.0 |
| Diisotearyl Fumarate | 12.0-25.0 | 12.0-25.0 |
| Oxetane-Substituted Chitosan Polyurethane | 5.0-15.0 | — |
| Metallo-Supramolecular Polymer | — | 5.0-15.0 |
| Petrolatum | 3.0-8.0 | 3.0-8.0 |
| Isopropyl Isotearate | 15.0-25.0 | 15.0-25.0 |
| Ozokerite | 0.1-3.0 | 0.1-3.0 |
| Candellila Wax | 0.1-3.0 | 0.1-3.0 |
| Tocopheryl Acetate | 0.1-3.0 | 0.1-3.0 |
| Titanium Dioxide | 0.1-3.0 | 0.1-3.0 |
| D&C Red No. 7 Ca. Lake | 0.2 | 0.2 |
| Red Iron Oxide | 0.3 | 0.3 |
| Pearls | 0.0-5.0 | 0.0-5.0 |
| Mica | 0.0-5.0 | 0.0-5.0 |
| Fragrance | 0.2 | 0.2 |

Anhydrous Cream-to-Powder Foundation

| Ingredients | Weight % | Weight % |
|---|---|---|
| Octyl Pamitate | 25.0-38.0 | 25.0-38.0 |
| Squalane | 15.0-25.0 | 15.0-25.0 |
| Oxetane-Substituted Chitosan Polyurethane | 5.0-15.0 | — |
| Metallo-Supramolecular Polymer | — | 5.0-15.0 |
| Beeswax | 2.0-5.0 | 2.0-5.0 |
| Microcrystalline Wax | 2.0-7.0 | 2.0-7.0 |
| Ozokerite | 2.0-7.0 | 2.0-7.0 |
| Polymethyl Methacrylate | 1.0-5.0 | 1.0-5.0 |
| Silica | 1.0-5.0 | 1.0-5.0 |
| Titanium Dioxide | 19.0 | 19.0 |
| Yellow Iron Oxide | 4.5 | 4.5 |
| Red Iron Oxide | 0.5 | 0.5 |
| Black Iron Oxide | 0.4 | 0.4 |
| Tocopheryl Acetate | 0.5 | 0.5 |

Longwear Liquid Foundation

| Ingredients | Weight % | Weight % |
|---|---|---|
| Cyclopentasiloxane | 8.0-15.0 | 8.0-15.0 |
| Dimethicone 100 cst | 2.0-5.0 | 2.0-5.0 |
| Cyclopentasiloxane/Dimethicone Copolyol | 12.0-18.0 | 12.0-18.0 |
| PEG-10 Dimethicone | 0.5-2.5 | 0.5-2.5 |
| Oxetane-Substituted Chitosan Polyurethane | 5.0-15.0 | — |
| Metallo-Supramolecular Polymer | — | 5.0-15.0 |
| Silica | 1.0 | 1.0 |
| Talc | 2.0 | 2.0 |
| Deionized Water | 28.0-42.0 | 28.0-42.0 |
| Propylene Glycol | 2.5-6.0 | 2.5-6.0 |
| Sodium Chloride | 0.5-1.5 | 0.5-1.5 |
| Methyl Paraben | 0.3 | 0.3 |
| Phenoxyethanol | 0.75 | 0.75 |
| Titanium Dioxide - Silicone Treated | 5.5 | 5.5 |
| Yellow Iron Oxide - Silicone Treated | 1.6 | 1.6 |
| Red Iron Oxide - Silicone Treated | 0.4 | 0.4 |
| Black Iron Oxide - Silicone Treated | 0.2 | 0.2 |

Mascara

| Ingredients | Weight % | Weight % |
|---|---|---|
| Isododecane | 20.0-30.0 | 20.0-30.0 |
| Hydrogenate Polyisobutene | 2.5-5.0 | 2.5-5.0 |
| Bentone Gel (Isododecane) | 2.0-6.0 | 2.0-6.0 |
| PEG/PPG-19/19 Dimethicone | 1.0-3.0 | 1.0-3.0 |
| Carnauba Wax | 3.0-6.5 | 3.0-6.5 |
| Ozokerite 170 | 3.0-6.5 | 3.0-6.5 |
| Microcrystalline Wax | 3.0-6.5 | 3.0-6.5 |
| Oxetane-Substituted Chitosan Polyurethane | 5.0-15.0 | — |
| Metallo-Supramolecular Polymer | — | 5.0-15.0 |
| Lanolin | 1.0-4.0 | 1.0-4.0 |
| Zinc Stearate | 1.0-4.0 | 1.0-4.0 |
| Deionized Water | 18.0-25.0 | 18.0-25.0 |
| Sodium Chloride | 0.5-1.5 | 0.5-1.5 |
| Methyl Paraben | 0.3 | 0.3 |
| Phenoxyethanol | 0.4 | 0.4 |
| Black Iron Oxide (Silicone Treated) | 9.8 | 9.8 |

Skin Care Lotion SPF
(Water in Silicone)

| Ingredients | Weight % | Weight % |
|---|---|---|
| Cyclopentasiloxane | 8.0-15.0 | 8.0-15.0 |
| Dimethicone 100 cst | 2.0-5.0 | 2.0-5.0 |
| Octyl Methoxycinnamate | 3.00 | 3.00 |
| Cyclopentasiloxane/Dimethicone Copolyol | 12.0-18.0 | 12.0-18.0 |
| PEG-10 Dimethicone | 0.5-2.5 | 0.5-2.5 |
| Micronized Titanium Dioxide | 4.0 | 4.0 |
| Oxetane-Substituted Chitosan Polyurethane | 5.0-15.0 | — |
| Metallo-Supramolecular Polymer | — | 5.0-15.0 |
| Silica | 1.0 | 1.0 |
| Deionized Water | 28.0-42.0 | 28.0-42.0 |
| Propylene Glycol | 2.5-6.0 | 2.5-6.0 |
| Sodium Chloride | 0.5-1.5 | 0.5-1.5 |
| Methyl Paraben | 0.3 | 0.3 |
| Phenoxyethanol | 0.75 | 0.75 |

Anti-Wrinkle Cream SPF

| Ingredients | Weight % | Weight % |
|---|---|---|
| Deionized Water | 45.35-60.35 | 45.35-60.35 |
| Glycerin | 3.00-5.00 | 3.00-5.00 |
| Xanthan gum | 0.20-0.50 | 0.20-0.50 |
| Disodium EDTA | 0.10 | 0.10 |
| Glycolic Acid (70%) | 3.00-5.00 | 3.00-5.00 |
| Ammonium Hydroxide | 0.9-1.5 | 0.9-1.5 |
| Methylparaben | 0.30 | 0.30 |
| C12-15 alkyl benzoate | 4.0-10.00 | 4.0-10.00 |
| Oxetane-Substituted Chitosan Polyurethane | 5.0-15.0 | — |
| Metallo-Supramolecular Polymer | — | 5.0-15.0 |
| Ethylhexyl methoxycinnamate | 6.00 | 6.00 |
| Benzophenone | 3.00 | 3.00 |
| Dimethicone 200 cst | 1.00-3.00 | 1.00-3.00 |
| Isopropyl myristate | 2.50-5.50 | 2.50-5.50 |
| BHT | 0.05 | 0.05 |
| Cetyl alcohol | 2.50-3.50 | 2.50-3.50 |
| Steareth-2 | 1.00-2.50 | 1.00-2.50 |
| Steareth-21 | 1.00-3.50 | 1.00-3.50 |
| Phenoxyethanol | 0.6 | 0.6 |

What is claimed is:

1. A cosmetic composition comprising one or more actives or adjuvant and from about 0.1 to about 60% by weight based on the weight of the composition of a self healing film forming polymer, wherein self healing of the self healing film forming polymer is initiated by absorption of UV light.

2. The cosmetic composition according to claim 1 comprising from about 1 to about 40% by weight based on the weight of the composition of the self healing film forming polymer.

3. The cosmetic composition according to claim 1 wherein the self-healing film forming polymer is a polyurethane comprising an oxetane substituted polysaccharide.

4. The cosmetic composition according to claim 3 wherein the self-healing film forming polymer is a polyurethane comprising an oxetane substituted chitosan polysaccharide.

5. The cosmetic composition according to claim 1 wherein the self-healing film forming polymer is a polyurethane comprising an oxetane substituted polysaccharide.

6. The cosmetic composition according to claim 1 wherein the self healing film forming polymer comprises a reaction product of: a) a polyol or a thiol; b) an isocyanate; and c) an oxetane substituted polysaccharide.

7. The cosmetic composition according to claim 6 wherein the isocyanate is selected from the group consisting of methylenediisocyanate, hexamethylenediisocyanate, isophorene diisocyanate, toluenediisocyanate, 2,4-toluene diisocyanate, 2,6-toluene diisocyanate, 2,2′-methylenediphenylene diisocyanate, 2,4′-methylenediphenylene diisocyanate, 4,4′-methylenediphenylene diisocyanate, polyphenylene polymethylene polyisocyanate, 2,4-methylcyclohexane diisocyanate, 2,6-methylcyclohexane diisocyanate, 2,2′-methylene dicyclohexylene diisocyanate, 2,4′-methylene dicyclohexylene diisocyanate, 4,4′-methylene dicyclohexylene diisocyanate, isophorone diisocyanate, 4-diisocyanatobutane, 1,5-diisocyanatopentane, 1,6-diisocyanatohexane, 1,4-cyclohexane diisocyanate and isomeric mixtures thereof.

8. The cosmetic composition according to claim 6 wherein the polyol is selected from the group consisting of ethylene glycol, diethylene glycol, triethylene glycol, tetraethylene glycol, polyethylene glycol, propylene glycol, dipropylene glycol, polypropylene glycol, 1,4-butanediol, 1,6-hexanediol, polytetramethylene glycol, and polyesterdiol.

9. The cosmetic composition according to claim 6 wherein the thiol is selected from the group consisting of pentaerythritol tetrakis(3-mercaptoproprionate), trifunctional thiols, tetrafunctional thiols, thiol esters, and thiol acrylates.

10. The cosmetic composition according to claim 6 wherein the oxetane substituted chitosan, pectin or, heparin is a oxetane substituted chitosan, pectin or heparin polysaccharide.

11. The cosmetic composition according to claim 7 wherein the oxetane substituted chitosan, pectin or, heparin is an oxetane substituted chitosan, pectin or heparin polysaccharide.

12. The cosmetic composition according to claim 8 wherein the oxetane substituted chitosan, pectin or, heparin is an oxetane substituted chitosan, pectin or heparin polysaccharide.

13. The cosmetic composition according to claim 8 wherein the isocyanate is selected from the group consisting of methylenediisocyanate, hexamethylenediisocyanate, isophorene diisocyanate, toluenediisocyanate, 2,4-toluene diisocyanate, 2,6-toluene diisocyanate, 2,2′-methylenediphenylene diisocyanate, 2,4′-methylenediphenylene diisocyanate, 4,4′-methylenediphenylene diisocyanate, polyphenylene polymethylene polyisocyanate, 2,4-methylcyclohexane diisocyanate, 2,6-methylcyclohexane diisocyanate, 2,2′-methylene dicyclohexylene diisocyanate, 2,4′-methylene dicyclohexylene diisocyanate, 4,4′-methylene dicyclohexylene diisocyanate, isophorone diisocyanate, 4-diisocyanatobutane, 1,5-diisocyanatopentane, 1,6-diisocyanatohexane, 1,4-cyclohexane diisocyanate and isomeric mixtures thereof and the oxetane substituted chitosan, pectin or, heparin is an oxetane substituted chitosan, pectin or heparin polysaccharide.

14. The cosmetic composition according to claim 13 wherein the oxetane substituted chitosan, pectin or, heparin is an oxetane substituted chitosan polysaccharide.

15. The cosmetic composition according to claim 1 wherein the self-healing film forming polymer is a metallo-supramolecular polymer.

16. The cosmetic composition according to claim 15 wherein the metallo-supramolecular polymer is prepared by treating a 2,6-bis(1-methyl benzamidazoyl)pyridine substituted ethylene/butylene co-oligomer with a Zn or La salt.

17. The cosmetic composition according to claim 16 wherein the Zn salt is Zn(NTf₂)₂ and the La(NTf₂)₃.

18. The cosmetic composition of claim 1 further comprising at least one additional ingredient selected from the group consisting of cosmetic, personal care, dermatological and pharmaceutical active agents.

19. The cosmetic composition of claim 19 comprising at least ingredient selected from the group consisting of colorants, fragrances, moisturizers, pH adjusters, fillers, emulsifying agents, emollients, sunscreens, retinoids, anti-acne agents, and skin protectants.

20. The cosmetic composition of claim 1, wherein said composition is a foundation, makeup, lip make-up, eyeliner, nail polish, mascara, skin lotion, sunscreen composition, shampoo or hair cosmetic conditioner.
